(19) United States
(12) Patent Application Publication
(10) Pub. No.: US 2023/0149267 A1
(43) Pub. Date: May 18, 2023

(54) READILY DISSOLVABLE CARTRIDGE COMPLEX AND INDUSTRIAL SYSTEMS AND PROCESSES OF FABRICATING THE SAME

(71) Applicant: CAPSULE MINIMAL LTD., Kibuts Ein Ha' Natziv (IL)

(72) Inventor: AMICHAI KRUPIK, Herzeliya (IL)

(73) Assignee: CAPSULE MINIMAL LTD., Kibuts Ein Ha' Natziv (IL)

(21) Appl. No.: 18/093,979

(22) Filed: Jan. 6, 2023

(30) Foreign Application Priority Data
Jul. 5, 2022 (IL) 294539

(51) Int. Cl. A61K 8/02; A61Q 19/10; A61Q 5/02; A61K 8/04; A61K 8/20

(52) U.S. Cl. CPC A61K 8/022; A61Q 19/10; A61Q 5/02; A61K 8/046; A61K 8/20; A61K 2800/222

(57) ABSTRACT

An industrial process of fabricating a readily dissolvable cartridge complex is described; a respective industrial manufacture system and a cartridge complex are further described; the process comprises: preparing an effervescent material, forming at least one segment of a segmental scaffolding, preparing a payload substance, forming structured payload substance pellets, accommodating the structured payload substance pellets in the compartments of the segmental scaffolding, compressing segment of the segmental scaffolding, iteratively forming a plurality of segments of the readily dissolvable cartridge complex; the industrial manufacture system comprises: at least one form, a segmental scaffolding fabricating machine, a structured payload substance pellets fabricating machine; the readily dissolvable cartridge complex comprises: a segmental scaffolding, a plurality of radial partitions of the segmental scaffolding, a plurality of segments of said dissolvable cartridge complex, axial partitions of the segmental scaffolding, a plurality of compartments and a plurality of structured payload pellets.

### BACKGROUND ART

It is believed that the current state of the art is represented by the following patent literature: U.S. Pat. Nos. 8,119,587, 8,476,211, 8,697,621, 8,980,816, 10,016,098, 10,485,739, 10,526,570, 10,894,005, 11,236,293, US2016101204, US2018216053, US2020102524, KR20130003570 and WO2021174183.

### TECHNICAL FIELD

In general, the present invention pertains to the art of industrial chemistry. In particular, the invention relates to a readily dissolvable cartridge complex as well as processes of fabricating the same.

Hygiene products industry, producing liquid soaps, body wash, hair care products and many other cosmetics is one of the most polluting. One unit of a liquid soap or shampoo product typically contains: a highly pollutive disposable plastic bottle, about 20 percent of active ingredients, some of which are environmental and health-harming and about 80 percent water. Most of the common production processes are environmentally harming, employing pollutive ships, trucks and warehouses, environmentally harmful labels and packaging materials. That unproportionable investment in packaging materials and water transportation is environmentally unsustainable.

Millions of liquid soap, shampoo and other hair or body care products in bottles are being sold every day around the world. Those plastic bottles require high energy consumption and other polluting inputs to be produced. Further to that, plastic bottles are to be disposed of after a short utilization. 245 million people in the United States used body wash in disposable bottles in 2020. According to research published by Statista Research department in 2021, approximately 1.5 billion disposable bottles of body wash are disposed every year, not including other products such as shampoos, conditioners, mouth wash, etc.

Plastic bottles production is one of the most pollutive industries and major environmental problems as of today. An environmental harming component, as of its initial production from fossil fuels, through its logistics and transportation and up to its disposal. Further to the direct pollution involved with transportation, there are many indirect mass transportation pollution processes, such as ships, trucks and containers manufacturing, packaging production, tiers, cranes, forklifts, etc. A direct pollutive process is the inefficient transport and storage of the empty plastic bottles (from the plastic factory to the soap factory and when disposed of after use), as a full trailer loaded with bottles will carry more than 95 percent air due to the bottles high volume. Warehouses involve highly pollutive direct and indirect processes. From giant electric power consuming warehouses built from pollutive construction materials, through logistic equipment such as cranes and forklifts to disposable packaging materials (stretch plastic, cardboard boxes, etc.), as well as fixed packaging and containing facilities (shelves, containers, etc.).

Disposing of plastic bottles is also one of the most harmful activities to the environment, polluting air, water and soil. Plastic bottles remain through hundreds of years, going through microplastic particles which are air and water carriable and virtually impossible to detect and/or clean. Disposed bottles also typically include active ingredient residues that further pollute soil and water sources. Moreover, each unit of a liquid soap or shampoo product typically contains packaging and labels. Plastic stretch films, cardboard cases, plastic, and paper labels are harmful to the environment in three main ways: direct and indirect pollutive production process; direct and indirect transportation production to and from the soap plant as well as to and the consumer, when disposed of; highly pollutive disposal, contaminating soil, water and air.

Moreover and perhaps more importantly, each unit of a liquid soap or shampoo product typically contains about 80 percent or more of water. Mixing and diluting hygiene product, with such a high percentage of water, enlarges their volume and causes massive pollution through their production and logistics as follows: mixing high amounts of water requires massive energy and machinery inputs in the production process for the mixing machines and conveying machinery, bottle filling machines, enormous storage spaces requirements, and internal logistics (forklifts, cranes, containers, shelves, etc.). Further to the direct environmentally harmful process, there are many highly pollutive processes involved with production and operation of equipment and machinery.

Almost all the efforts and inputs, involved with the product transportation, are actually devoted to transportation of water. The water handling effect includes a direct pollutive process is grossly inefficient transport of water for thousands of miles each time by highly pollutive vessels such as ships, trains, and trucks along with all collateral logistic equipment and containers. Additional indirect pollution is caused by the peripheral industries involved, such as trucks, ships and various equipment production industries. 95 percent and more of the environmental harming processes are dedicated to simply manipulation of water. The storage of water in warehouses and stores causes massive direct and indirect environmental harm. From giant electric power consuming warehouses built from pollutive construction materials, through logistic equipment such as cranes and forklifts to disposable packaging materials (stretch plastic, cardboard boxes, etc.) and fixed containing facilities (shelves, containers, etc.) packaging and.

Therefore liquid soap, shampoo and most of all other hygiene and self-care products in use are harming the environment and contribute to unproportioned environmental abuse.

### SUMMARY OF THE INVENTION

The following summary of the invention is provided in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

The invention was made in view of the deficiencies of the prior art and provides systems, methods and processes for overcoming these deficiencies. According to some embodiments and aspects of the present invention, there is provided an eco-friendly, healthy and economical care hygiene product, including a readily dissolvable water-soluble cartridge complex, containing all required active ingredients of the product. Therefore, instead of purchasing a bottle filled with the product, the user buys a solid readily dissolvable cartridge complex with a volume of only up to 20 percent of the ordinary product, puts the readily dissolvable cartridge complex in a reusable bottle, fills the bottle with water from the tap and in a short time receives a high-quality finished product, in a non-limiting manner including: shampoo, body soap, hand soap, conditioner, floor cleaner, dishwashing soap, home cleaning products, mouthwash and/or any other liquid with substantial water content.

The readily dissolvable cartridge complex, along with the savings of unnecessary water transportation, logistics and all the packaging material, provides an eco-friendly product, for less money than spent today on ordinary environmentally harming products.

In accordance with some aspects and embodiments of the present invention there is provided a readily dissolvable cartridge complex including: a segmental scaffolding including an effervescent material readily dissolvable in an aqueous solution; a plurality of radial partitions of the segmental scaffolding, arranged in tandem, extending about the longitudinal centerline of the dissolvable cartridge complex; a plurality of segments of the dissolvable cartridge complex, separated by the radial partitions and formed in-between the radial partitions; a plurality of axial partitions of the segmental scaffolding, extending transversally to and along the longitudinal centerline of the dissolvable cartridge complex, dividing each one of the plurality of the segments of the dissolvable cartridge complex into a plurality of sectors; a plurality of compartments formed in-between the axial partitions within the sectors, in each one of the plurality of the segments of the dissolvable cartridge complex; a plurality of structured payload pellets, accommodated in the compartments.

In some embodiments the dissolvable cartridge complex includes a coating configured to absorb and/or neutralize undesired additives or impurities in water.

In some embodiments the dissolvable cartridge complex includes a coating including a water softening agent.

In some embodiments the dissolvable cartridge complex includes a coating including natrium chloride.

In accordance with some aspects and embodiments of the present invention there is provided an industrial process of fabricating a readily dissolvable cartridge complex including: preparing an effervescent material readily dissolvable in an aqueous solution; forming at least one segment of a segmental scaffolding from the effervescent material including: forming at least on radial partition of the at least one segment of the segmental scaffolding, extending about the longitudinal centerline of the dissolvable cartridge complex; forming a plurality of axial partitions within the at least one segment of the segmental scaffolding, extending transversally to and along the longitudinal centerline of the dissolvable cartridge complex; forming a plurality of compartments formed in-between the axial partitions, in each one of the plurality of the segments of the segmental scaffolding; preparing a payload substance; forming a plurality of structured payload substance pellets; accommodating the structured payload substance pellets in the compartments of the segmental scaffolding; compressing at least one segment of the segmental scaffolding of the readily dissolvable cartridge complex; iteratively forming a plurality of segments of the readily dissolvable cartridge complex separated by the radial partitions.

In some embodiments the industrial process further includes furnishing the readily dissolvable cartridge complex with a coating configured to absorb and/or neutralize undesired additives or impurities in water.

In some embodiments the industrial process further includes furnishing the readily dissolvable cartridge complex with a coating including water softening agent, including natrium chloride.

In some embodiments the industrial process further includes controllably saturating at least one member selected from the group consisting of: the payload substance and the effervescent material, with a predefined amount of saturating substrate, thereby conferring to the at least one member a semi-liquefied consistency.

In some embodiments the industrial process further includes deploying at least one stencil, in which an exterior surface of the at least one stencil essentially conforms and/or matches with at least one shape selected from the group consisting of: a shape of axial partitions of the segmental scaffolding, extending transversally to and along the longitudinal centerline of the dissolvable cartridge complex and a shape of the structured payload substance pellets.

In some embodiments the compressing of the industrial process further includes essentially conforming and/or matching with exterior outline of a shape of the structured payload substance pellets.

In accordance with some aspects and embodiments of the present invention there is provided an industrial manufacture system for fabricating a readily dissolvable cartridge complex includes: at least one form, including an essentially hollow shape, in which an interior surface of the at least one form essentially conforms and/or matches with a shape of the readily dissolvable cartridge complex; a segmental scaffolding fabricating machine including: an effervescent material reservoir, containing an effervescent material readily dissolvable in an aqueous solution; a dossing module connected to the reservoir of effervescent material, configured to receive the effervescent material from the reservoir of effervescent material and to portion a predetermined amount of the effervescent material; a fabricating module including at least one nozzle, configured to dispose the effervescent material in a predefined structured arrangement within the form, thereby forming a plurality of compartments, within at least one segment of the segmental scaffolding; a compacting mechanism, including at least one pressing piston operationally connected to a pressure exerting device, configured to compress at least one segment of the segmental scaffolding of the readily dissolvable cartridge complex within the form; a structured payload substance pellets fabricating machine including: a payload substance reservoir, containing a payload substance; a dossing module connected to the reservoir of payload substance, configured to receive the payload substance from the reservoir of payload substance and to portion a predetermined amount of the payload substance; a fabricating module including at least one nozzle, configured to dispose the payload substance in a predefined structured arrangement within the form, thereby forming a plurality of structured payload substance pellets within the compartments in at least one segment of the segmental scaffolding; a compacting mechanism, including at least one pressing piston operationally connected to a pressure exerting device, configured to compress the structured payload substance pellets within the compartments, thereby accommodating the structured payload substance pellets in the compartments, formed within at least one segment of the segmental scaffolding of the readily dissolvable cartridge complex, within the form.

In some embodiments the industrial manufacture system further includes a saturating module, configured for controllably saturate at least one member selected from the group consisting of: the payload substance and the effervescent material, with a predefined amount of saturating substrate, thereby conferring to the at least one member a semi-liquefied consistency.

In some embodiments the industrial manufacture system further includes a pulverizator, configured for furnishing the readily dissolvable cartridge complex with a coating configured to absorb and/or neutralize undesired additives or impurities in water.

In some embodiments the industrial manufacture system further includes at least one stencil, in which an exterior surface of the at least one stencil essentially conforms and/or matches with at least one shape selected from the group consisting of: a shape of axial partitions of the segmental scaffolding, extending transversally to and along the longitudinal centerline of the dissolvable cartridge complex; a shape of the structured payload substance pellets.

In some embodiments the compacting mechanism of the structured payload substance pellets fabricating machine includes a plurality of pressing pistons, in which an exterior outline of the pistons essentially conforms and/or matches with exterior outline of a shape of the structured payload substance pellets.

### DEFINITIONS

The term structured as referred to herein is to be construed as including any geometrical shape, exceeding in complexity a plain linear shape or a shape embodying simple cylindrical, elliptical or polygonal contour or profile. A more complex shape, a plain linear shape or a shape embodying simple cylindrical, elliptical or polygonal contour or profile, constitutes an example of structured geometry.

The term effervescent material as referred to herein is to be construed as including any material, mix of materials and/or composition capable of effervescence upon contact with water. Effervescence, exemplified in this context, can mean the emission of gas bubbles from a liquid as a result of a chemical reaction between a soluble acid source and an alkali metal carbonate to produce carbon dioxide gas.

The term payload substance as referred to herein is to be construed as including any material, mix of materials and/or composition including an anhydrous or essentially dry concentrated form and/or concentrate of any hygiene products, such as liquid soaps, body wash, hair care products and any other cosmetics, as well as any other household and/or hygiene products. The term payload substance as referred to herein can be construed as including any active ingredient of any household and/or hygiene product.

The terms method and process as used herein are to be construed as including any sequence of steps or constituent actions, regardless a specific timeline for the performance thereof. The particular steps or constituent actions of any given method or process are not necessarily in the order they are presented in the claims, description or flowcharts in the drawings, unless the context clearly dictates otherwise. Any particular step or constituent action included in a given method or process may precede or follow any other particular step or constituent action in such method or process, unless the context clearly dictates otherwise. Any particular step or constituent action and/or a combination thereof in any method or process may be performed iteratively, before or after any other particular step or action in such method or process, unless the context clearly dictates otherwise. Moreover, some steps or constituent actions and/or a combination thereof may be combined, performed together, performed concomitantly and/or simultaneously and/or in parallel, unless the context clearly dictates otherwise. Moreover, some steps or constituent actions and/or a combination thereof in any given method or process may be skipped, omitted, spared and/or opted out, unless the context clearly dictates otherwise.

The term modular, as referred to herein, should be construed as a stand-alone unit. The term modular inter alia means a standardized unit that may be conveniently installed or deployed without significant impact to the environment. The term modular, however, doesn't necessarily mean providing for ease of interchange or replacement. The term modular is optionally satisfied by providing for ease of at least onetime deployment or installation.

The term readily connectable, as referred to herein, should be construed as a standardized unit that may be conveniently connected to other components of the system. The term readily connectable, however, doesn't necessarily mean readily disconnectable or removable. The term readily connectable is optionally satisfied by providing for ease of at least onetime connection or coupling.

By operationally connected and operably coupled or similar terms used herein is meant connected in a specific way (e.g., in a manner allowing fluid to move and/or electric power to be transmitted) that allows the disclosed system and its various components to operate effectively in the manner described herein.

The term fluid or liquid as referred to herein is to be construed as any material that deforms when a shear stress is applied. While fluid generally would refer to any liquids or gases, it may be used herein to describe fluidized solids and bulk solids and/or granulate matter that are capable of flowing or otherwise moving inside a device as a result of pressure differences and/or gravitational force. Such materials may include slurries, suspensions, pastes, powders, granular solids, particle solids, granulate matter, particulate matter, as well as any combinations thereof.

The term slurry, as referred to herein, is to be construed as a mixture of solids denser than water suspended in liquid, usually water. Solids concentrations in a slurry typically range between about 0.5 percent and about 5 percent.

The term sludge, as referred to herein, is to be construed as a semi-solid slurry. The term is also sometimes used as a generic term denoting solids separated from suspension in a liquid. Solids concentrations in a sludge typically range between about 5 percent and about 15 percent.

In the specification or claims herein, any term signifying an action or operation, such as: a verb, whether in base form or any tense, gerund or present/past participle, is not to be construed as necessarily to be actually performed but rather in a constructive manner, namely as to be performed merely optionally or potentially.

The term substantially as used herein is a broad term, and is to be given its ordinary and customary meaning to a person of ordinary skill in the art (and is not to be limited to a special or customized meaning), and refers without limitation to being largely but not necessarily entirely of that quantity or quality which is specified.

The term essentially means that the composition, method or structure may include additional ingredients, stages and or parts, but only if the additional ingredients, the stages and/or the parts do not materially alter the basic and new characteristics of the composition, method or structure claimed.

As used herein, the term essentially changes a specific meaning, meaning an interval of plus or minus ten percent (±10 percent). For any embodiments disclosed herein, any disclosure of a particular value, in some alternative embodiments, is to be understood as disclosing an interval approximately or about equal to that particular value (i.e., ±10 percent).

As used herein, the terms about or approximately modify a particular value, by referring to a range equal to the particular value, plus or minus twenty percent (+/−20 percent). For any of the embodiments, disclosed herein, any disclosure of a particular value, can, in various alternate embodiments, also be understood as a disclosure of a range equal to about that particular value (i.e. +/−20 percent).

As used herein, the term or is an inclusive or operator, equivalent to the term and/or, unless the context clearly dictates otherwise; whereas the term and as used herein is also the alternative operator equivalent to the term and/or, unless the context clearly dictates otherwise.

It should be understood, however, that neither the briefly synopsized summary nor particular definitions hereinabove are not to limit interpretation of the invention to the specific forms and examples but rather on the contrary are to cover all modifications, equivalents and alternatives falling within the scope of the invention.

### DESCRIPTION OF THE DRAWINGS

The present invention will be understood and appreciated more comprehensively from the following detailed description taken in conjunction with the appended drawings in which:

FIG. 1A is a perspective view of a readily dissolvable cartridge complex, according to some embodiments of the present invention;

FIG. 1B is an enlarged perspective view of the readily dissolvable cartridge complex, according to some embodiments of the present invention;

FIG. 2A is a top view of a segment of the readily dissolvable cartridge complex, according to some embodiments of the present invention;

FIG. 2B is a perspective view of a segment of the readily dissolvable cartridge complex, according to some embodiments of the present invention;

FIG. 3A is an isometric view of a readily dissolvable cartridge complex, according to some embodiments of the present invention;

FIG. 3B is a top view of a segment of the readily dissolvable cartridge complex, according to some embodiments of the present invention;

FIG. 3C is a perspective view of a compartment of a segment of the readily dissolvable cartridge complex, according to some embodiments of the present invention;

FIG. 4A is a perspective view of a reusable dispenser bottle into which a readily dissolvable cartridge complex is introduced, according to some embodiments of the present invention;

FIG. 4B is an exploded view of a reusable dispenser bottle into which dissolvable cartridge complex is introduced, according to some embodiments of the present invention;

FIG. 4C is an isometric view of plurality of cups of plurality of reusable dispenser bottles in a nested and stacked configuration, according to some embodiments of the present invention;

FIG. 4D is a side view of a cover of a reusable dispenser bottle, according to some embodiments of the present invention;

FIG. 4E is a top view of a cover of a reusable dispenser bottle, in a closed configuration, according to some embodiments of the present invention;

FIG. 4F is a top view of a cover of a reusable dispenser bottle, in an open configuration, according to some embodiments of the present invention;

FIG. 5 is a block diagram of an industrial manufacture system for fabricating a readily dissolvable cartridge complex, according to some embodiments of the present invention;

FIG. 6A is an exploded view of a form and compacting mechanism complex including a stencil of an industrial manufacture system for dry fabricating of a readily dissolvable cartridge complex, according to some embodiments of the present invention;

FIG. 6B is a schematic view of a form and compacting mechanism complex including a stencil of an industrial manufacture system for dry fabricating of a readily dissolvable cartridge complex, according to some embodiments of the present invention, showing the stencil within the form;

FIG. 7A is an exploded view of a form and compacting mechanism complex including a structured nozzle of an industrial manufacture system for fabricating of a readily dissolvable cartridge complex, according to some other embodiments of the present invention;

FIG. 7B is a schematic view of a structured nozzle introduced into the form, according to some other embodiments of the present invention;

FIG. 7C is a schematic view of the formation of one segment of the readily dissolvable cartridge complex, according to some other embodiments of the present invention;

FIG. 7D is a schematic view of the formation of a radial partition of the readily dissolvable cartridge complex, according to some other embodiments of the present invention;

FIG. 7E is a schematic view of the readily dissolvable cartridge complex formed within the form, according to other embodiments of the present invention;

FIG. 7F is an exploded view of the readily dissolvable cartridge complex formed within the form and a pressing array, according to some other embodiments of the present invention;

FIG. 7G is a schematic view of a pressing piston compacting the readily dissolvable cartridge complex within the form, according to some other embodiments of the present invention;

FIG. 7H is a schematic view of a pressing piston ejecting the readily dissolvable cartridge complex from the form, according to some other embodiments of the present invention;

FIG. 7I is a schematic view of a pressing piston withdrawn into the from after the readily dissolvable cartridge complex was ejected from, according to some other embodiments of the present invention;

FIG. 8 is a flowchart of an industrial process of fabricating a readily dissolvable cartridge, according to some embodiments of the present invention;

FIG. 9 is a flowchart of an industrial process of essentially dry fabricating a readily dissolvable cartridge, according to some embodiments of the present invention;

FIG. 10 is a flowchart of an industrial process of essentially dry fabricating a readily dissolvable cartridge, according to some other embodiments of the present invention.

While the invention is susceptible to various modifications and alternative forms, specific embodiments thereof have been shown merely by way of example in the drawings. The drawings are not necessarily complete and components are not essentially to scale; emphasis instead being placed upon clearly illustrating the principles underlying the present invention.

### DETAILED DISCLOSURE OF EMBODIMENTS

Illustrative embodiments of the invention are described below. In the interest of clarity, not all features of actual implementation are described in this specification. It should be appreciated that various features or elements described in the context of some embodiment may be interchangeable with features or elements of any other embodiment described in the specification. Moreover, it will be appreciated that for the development of any actual embodiment, numerous implementation-specific decisions must be made to achieve the developers' specific goals, such as compliance with technology- or business-related constraints, which may vary from one implementation to another, and the effort of such a development might be complex and time-consuming, but would nevertheless be a routine undertaking for those of ordinary skill in the art having the benefit of this disclosure.

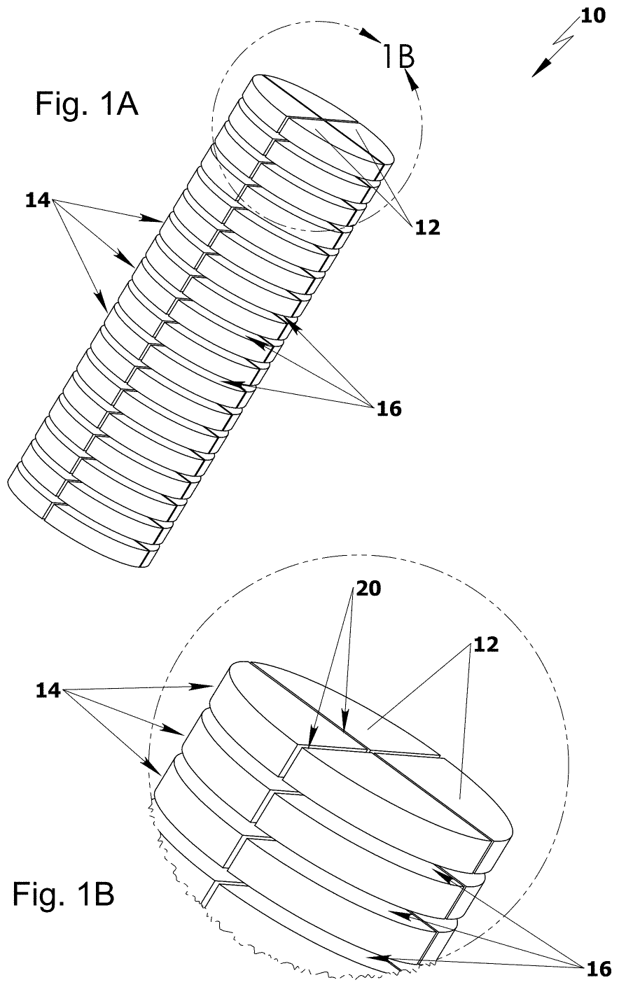

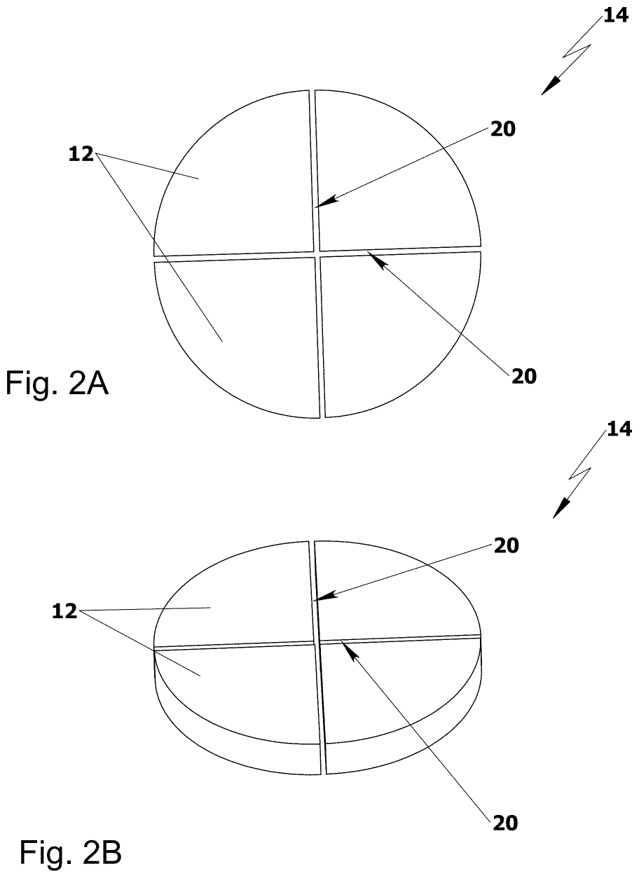

In accordance with some embodiments of the present invention, reference is now made to FIG. 1A to 3C, showing readily dissolvable cartridge complex 10. In some embodiments, readily dissolvable cartridge complex 10 comprises a plurality of structured payload substance pellets 12, embedded within individual segments 14 of a segmental scaffolding. Segments 14 of segmental scaffolding comprises an effervescent material readily dissolvable in an aqueous solution.

The effervescent material of the segmental scaffolding may be selected from appropriate material which exhibits effervesce on contact with water. In one example, the effervescent material comprises at least sodium bicarbonate and citric acid. These materials are present in any suitable amounts to achieve effervescence. One skilled in the art is able to combine these materials to provide the desired rate of effervescence.

In other examples, the effervescent material comprises sodium bicarbonate in an amount of 40 to 75 wt percent. In other examples, the effervescent material comprises sodium bicarbonate in an amount of 50 to 70 wt percent. In other examples, the effervescent material comprises sodium bicarbonate in an amount of 55 to 70 wt percent. In some examples, the effervescent material comprises citric acid in an amount of 24 to 40 wt percent. In other examples, the effervescent material comprises citric acid in an amount of 26 to 38 wt percent based on the first effervescent material. In other examples, the effervescent material comprises citric acid in an amount of 28 to 36 wt percent. In other examples, the effervescent material comprises citric acid in an amount of 30 to 34 wt percent. In some examples, the effervescent material comprises sodium bicarbonate in an amount of 40 to 75 wt percent and citric acid in an amount of 24 to 40 wt percent.

In some embodiments, pellets 12 comprise a payload substance, including a surfactant in an amount of 0.5 to 5 wt percent. The surfactant of the payload substance is primarily selected from those surfactants known in the art to be suitable for contact with the skin. In one embodiment, the surfactant is selected from the group consisting of sodium laureth sulfate, cocamide diethanolamine, lauryl betaine and mixtures thereof. In one embodiment, the surfactant is sodium laureth sulfate.

In some embodiments, the surfactant of the payload substance provides the composition with the ability to achieve its required purpose. Thus for a cleanser, the surfactant creates foam and removes dirt and grease from the user's skin.

In some embodiments, payload substance pellets 12 further comprise a fragrance. Preferably the fragrance is present in an amount of 0.5 to 4 wt percent. In some embodiments, payload substance pellets 12 comprise fragrance in an amount of 1 to 4 wt percent. In some embodiments, payload substance pellets 12 comprise fragrance in an amount of 2 to 4 wt percent. In some embodiments, payload substance pellets 12 comprise fragrance in an amount of 2.5 to 3.5 wt percent.

In some embodiments, payload substance pellets 12 further comprise another material, whether soluble or insoluble, which us incorporated into the product to provide further effects, for example with regard to odor, taste, pH, texture colour or foam production and etc.

In some embodiments, readily dissolvable cartridge complex 10 further comprises a plurality of radial partitions 16 of segmental scaffolding. Radial partitions 16 are arranged in tandem, extending about a longitudinal centerline of dissolvable cartridge complex 10. Radial partitions 16 divide adjacent segments 14 of the segmental scaffolding. In some embodiments, readily dissolvable cartridge complex 10 comprises plurality of segments 14. Segments 14 are separated by radial partitions 16 and formed in-between radial partitions 16.

In some embodiments, readily dissolvable cartridge complex 10 comprises plurality of axial partitions 20 of the segmental scaffolding. Axial partitions 20 extend transversally to and along the longitudinal centerline of dissolvable cartridge complex 10, dividing each one of plurality of segments 14.

In some embodiments, the segmental scaffolding of readily dissolvable cartridge complex 10 further comprises a plurality of compartments. The compartments are formed in-between axial partitions 20 of the segmental scaffolding, in each one of plurality of segments 14 of the segmental scaffolding. In some embodiments, readily dissolvable cartridge complex 10 comprises a plurality of structured payload pellets 12. Structured payload pellets 12 are accommodated in the compartments of the segmental scaffolding.

In some embodiments, dissolvable cartridge complex 10 is configured for rapidly or even quote instantly providing for an extensive surface area of structured payload pellets 12, upon contact with water. After the segmental scaffolding undergoes rapid or even quote instant effervescence, the accumulative surplus surface area of all structured payload pellets 12 accelerates the solubility and increasing mixing and molecule diffusion rate, by removing radial partitions 16 and axial partitions 20 of the segmental scaffolding.

In some embodiments, dissolvable cartridge complex 10 is coated by dissoluble coating 24. When dissolvable cartridge complex 10 is placed in water or other essentially aqueous solution, dissoluble coating 24 rapidly or even quote dissolves in water. Then uncoated dissolvable cartridge complex 10 is exposed to water thereby initiating an effervescence reaction. Dissoluble coating 24 is configured to modify the water or other aqueous solution, so as to sequester, absorb, precipitate, balance or otherwise neutralize undesired impurities or additives in the water.

In some examples, dissoluble coating 24 comprising a water softening agent, such as natrium chloride, configured to neutralize water hardening additives in the water, such as calcium and magnesium ions. In other examples, dissoluble coating 24 comprising a sequestering agent, such as EDTA or other chelate forming agent, configured to sequester metal ions in aqueous solution. In yet other examples, dissoluble coating 24 comprising a disinfectant, such as an oxidating or bleaching agent, configured to neutralize biological impurities in the water, for instance: iodine, chlorine, chloramine, chlorine and dioxide, and/or malodor treatment.

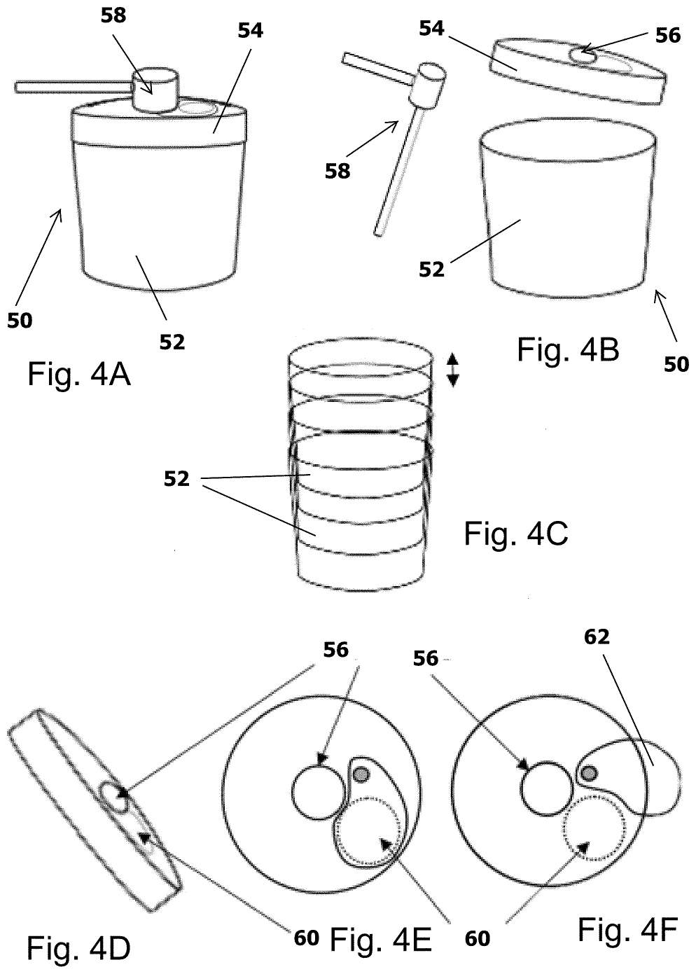

In accordance with some embodiments of the present invention, reference is now made FIG. 4A to 4F showing reusable dispenser 50 into which dissolvable cartridge complex 10, shown in FIG. 1A to 3C and described hereinabove, is administrated. The reusable dispenser of the embodiment of FIG. 4A to 4F illustrates various features that may be interchangeable with elements of any other embodiment described in the specification.

In some embodiments, reusable dispenser 50 comprises container 52. Container 52 is configured for receiving the dissolvable cartridge complex, such as dissolvable cartridge complex 10 shown in FIG. 1A to 3C. In some embodiments, container 52 is covered by cover 54. Cover 54 preferably comprises aperture 56. Aperture 56 is typically configured for accommodating dispensing pump 58.

In some examples, dispensing pump 58 is of the type which is pressed with manual force to dispense the liquid. In other examples, dispensing pump 58 is of the electromechanical type, coupled to a power source and driven by electric power, to perform the dispensing action. In some examples, electromechanical dispensing pump 58 is manually activated upon physical contact with an electric switch, knob or button, whereas in other examples electromechanical dispensing pump 58 is configured for contactless activation, typically by an IR detector. In some examples, dispensing pump 58 comprises a foam dispenser, comprising a mechanism configured to transform the liquid dispensed from reusable dispenser 50 into a light or thick foam.

In some embodiments, cover 54 of reusable dispenser 50 further comprises cartridge aperture 60. The dissolvable cartridge complex, such as dissolvable cartridge complex 10 shown in FIG. 1A to 3C, is introduced into container 52 through cartridge aperture 60. Cartridge aperture 60 is optionally coverable by lid 62. Preferably, lid 62 is operationally connected to cover 54. It should be acknowledged that the configuration of reusable dispenser 50 with cover 54, with cover on top, with cartridge aperture 60 is merely exemplary, whereas any other configuration is equally applicable.

In some embodiments reusable dispenser bottle 50 is eco-efficient and reusable as well as recyclable. In some examples, reusable dispenser bottle 50 is made of stainless steel and/or recycled aluminum and/or any other material or composition. In other embodiments, reusable dispenser bottle 50 is disassembled so that a plurality of containers 52 and plurality of covers 54 are nested in a stack one on top of another, such as shown in FIG. 4C, thereby reducing shipping volume of dispensers 50.

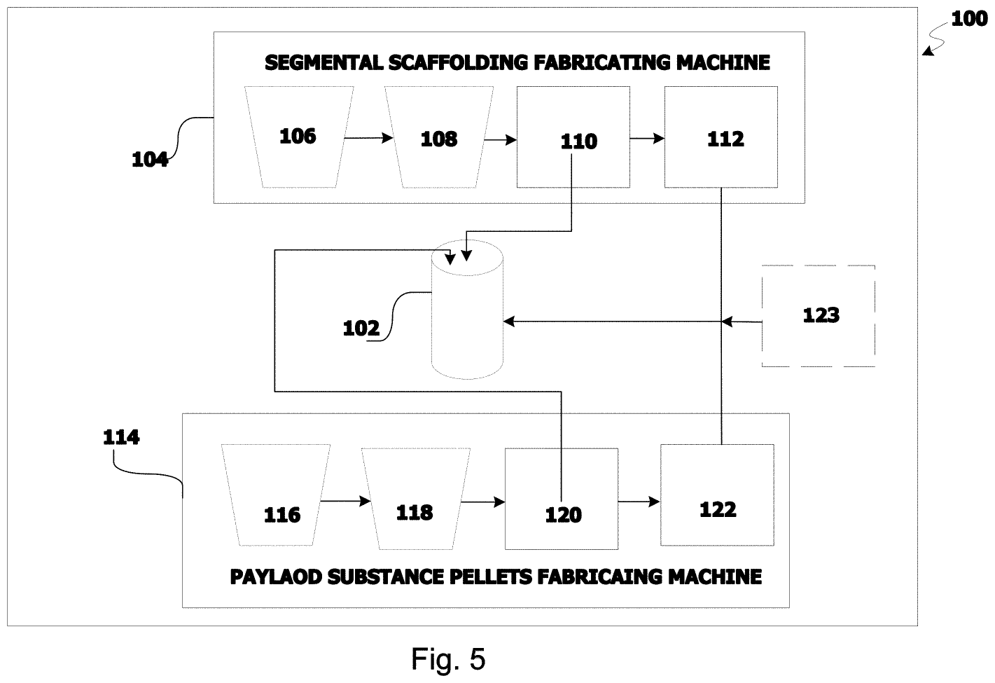

In accordance with some embodiments of the present invention, reference is now made to FIG. 5, showing industrial manufacture system 100 for fabricating a readily dissolvable cartridge complex. Industrial manufacture system 100 of the embodiment of FIG. 5 illustrates various features that may be interchangeable with elements of any other embodiment described in the specification.

In some embodiments, system 100 for fabricating a readily dissolvable cartridge complex comprises at least one form 102. Form 102 comprising an essentially hollow shape, in which an interior surface of at least one form 102 essentially conforms and/or matches with a shape of dissolvable cartridge complex, such as dissolvable cartridge complex 10 shown in FIG. 1A to 3C and described hereinabove.

In some embodiments, system 100 further comprises segmental scaffolding fabricating machine 104. In some embodiments, segmental scaffolding fabricating machine 104 comprises effervescent material reservoir 106. Effervescent material reservoir 106 contains and stores an effervescent material readily dissolvable in an aqueous solution. In some examples, effervescent material reservoir 106 comprises a conical stainless steel reservoir or a miniature silo. In some embodiments, the effervescent material effervescent material in effervescent material reservoir 106 is typically in the form of dry powder. The term dry powder, as referred to herein, may include any substance comprising one or a plurality of constituents or ingredients with one or a plurality of (average) particulate size ranges.

In some embodiments, segmental scaffolding fabricating machine 104 comprises dosing module 108. Dosing module 108 is connected to effervescent material reservoir 106 and configured to receive the effervescent material from effervescent material reservoir 106. Dosing module 108 is configured to portion a predetermined amount of the effervescent material.

In some examples, dosing module 108 comprises a predefined volume and/or a weighting scale device. Dosing module 108 is configured to portion the effervescent material of various predetermined amounts, volumes and or weights. From many kilograms and bulk volume of effervescent material in reservoir 106, dosing module 108 portions relatively small and predetermined amounts.

In some embodiments, segmental scaffolding fabricating machine 104 comprises fabricating module 110. Fabricating module 110 comprises at least one nozzle, configured to dispose the effervescent material in a predefined structured arrangement within form 102, thereby forming a plurality of compartments, within at least one segment of the segmental scaffolding. In some preferred embodiments, fabricating module 110 comprises at least one nozzle, configured to dispose the effervescent material in a predefined location and/or coordinate within form 102 in a controllable manner, somewhat similarly to a jet printer, thereby gradually forming the radial and axial portions step-by-step, of at least one segment of the segmental scaffolding.

In some embodiments, segmental scaffolding fabricating machine 104 comprises compacting mechanism 112. Compacting mechanism 112 comprises at least one pressing piston operationally connected to pressure exerting device 123, configured to compress at least one segment of the readily dissolvable cartridge complex within form 102. In some embodiments, when the effervescent material is compressed, it undergoes agglomeration and is capable thereafter to inherently maintain the structural integrity of the readily dissolvable cartridge complex.

In some embodiments, system 100 is configured for semi-liquefied fabricating of the readily dissolvable cartridge complex. In such embodiments, segmental scaffolding fabricating machine 104 of system 100 further comprises a saturating module, configured for controllably saturating the effervescent material, with a predefined amount of saturating substrate, thereby conferring to the effervescent material a semi-liquefied consistency. In some examples, the saturating module comprises a substrate storage unit and/or a substrate dosimeter. The substrate storage unit is configured storing the substrate or liquid added to the effervescent material, whereas the substrate dosimeter is configured for controllably adding a certain amount of substrate or liquid to the effervescent material, thereby conferring to the effervescent material a semi-liquefied consistency.

In some embodiments, segmental scaffolding fabricating machine 104 comprises a blender. The blender is configured for mixing the dosed saturating substrate or liquid with a predefined amount of effervescent material, thereby conferring homogeneity to the effervescent material, optionally with semi-liquefied consistency.

In some embodiments, system 100 further comprises structured payload substance pellets fabricating machine 114. In some embodiments, structured payload substance pellets fabricating machine 114 comprises payload substance reservoir 116. In some embodiments, the payload substance in payload substance reservoir 116 is typically in the form of dry powder. Payload substance reservoir 116 contains and stores a payload substance. In some examples, payload substance reservoir 116 comprises a conical stainless steel reservoir or a miniature silo.

In some embodiments, structured payload substance pellets fabricating machine 114 further comprises dosing module 118. Dosing module 118 is connected to payload substance reservoir 116 and configured to receive the payload substance from reservoir 116. Dosing module 118 is configured to portion a predetermined amount of the payload substance. Dosing module 108 is configured to portion a predetermined amount of the payload substance.

In some embodiments, structured payload substance pellets fabricating machine 114 comprises fabricating module 120. In some embodiments, payload substance pellets fabricating module 120 comprises at least one nozzle, configured to dispose the payload substance, optionally having a semi-liquefied consistency. In such embodiments, the at least one nozzle is configured to dispose the payload substance in a predefined structured arrangement within form 102, thereby forming a plurality of structured payload substance pellets within the compartments of the segmental scaffolding, in at least one segment of the of the readily dissolvable cartridge complex. In some preferred embodiments, fabricating module 120 comprises at least one nozzle, configured to dispose the payload substance in a predefined location and/or coordinate within form 102 in a controllable manner, somewhat similarly to a jet printer, thereby gradually forming the structured payload substance pellets step-by-step, of at least one segment of the readily dissolvable cartridge complex.

In some embodiments, the at least one nozzle of fabricating module 110 configured to dispose the effervescent material in a predefined location and/or coordinate within form 102, as well as the at least one nozzle of fabricating module 120 configured to dispose the payload substance in a predefined location and/or coordinate within form 102, in a controllable manner somewhat similarly to a jet printer, thereby gradually forming step-by-step the segment of the segmental scaffolding, are exchangeable nozzles on a same disposing machine, such as an extruder, dispenser, press, pump or ejector.

Fabricating module 110 comprises at least one nozzle, configured to dispose the effervescent material in a predefined structured arrangement within form 102, thereby forming a plurality of compartments, within at least one segment of the segmental scaffolding. In some preferred embodiments, fabricating module 110 comprises at least one nozzle, configured to dispose the effervescent material in a predefined location and/or coordinate within form 102 in a controllable manner, somewhat similarly to a jet printer, thereby gradually forming the radial and axial portions step-by-step, of at least one segment of the segmental scaffolding.

In some preferred embodiments, fabricating module 120 comprises at least one structured nozzle, for instance embodying the shape of the segmental scaffolding and/or of the structured payload substance pellets and/or any portion thereof. A structured nozzle optionally disposes an entire fragment of at least one segment of the readily dissolvable cartridge complex, at once.

In some embodiments, structured payload substance pellets fabricating machine 114 further comprises compacting mechanism 122. Compacting mechanism 122 comprises at least one pressing piston operationally connected to pressure exerting device 123, configured to compress the structured payload substance pellets within the compartments of the segmental scaffolding, thereby accommodating the structured payload substance pellets in the compartments, formed within at least one segment of the segmental scaffolding of the readily dissolvable cartridge complex, within form 102.

In some embodiments, system 100 is configured for semi-liquefied fabricating of the readily dissolvable cartridge complex. In such embodiments, structured payload substance pellets fabricating machine 114 of system 100 further comprises a saturating module, configured for controllably saturating the payload substance, with a predefined amount of saturating substrate, thereby conferring to the payload substance a semi-liquefied consistency. In some examples, the saturating module comprises a substrate storage unit and/or a substrate dosimeter. The substrate storage unit is configured storing the substrate or liquid added to the payload substance, whereas the substrate dosimeter is configured for controllably adding a certain amount of substrate or liquid to the payload substance, thereby conferring to the payload substance a semi-liquefied consistency.

In some embodiments, structured payload substance pellets fabricating machine 114 comprises a blender. The blender is configured for mixing the dosed saturating substrate or liquid with a predefined amount of payload substance, thereby conferring homogeneity to the payload substance with semi-liquefied consistency.

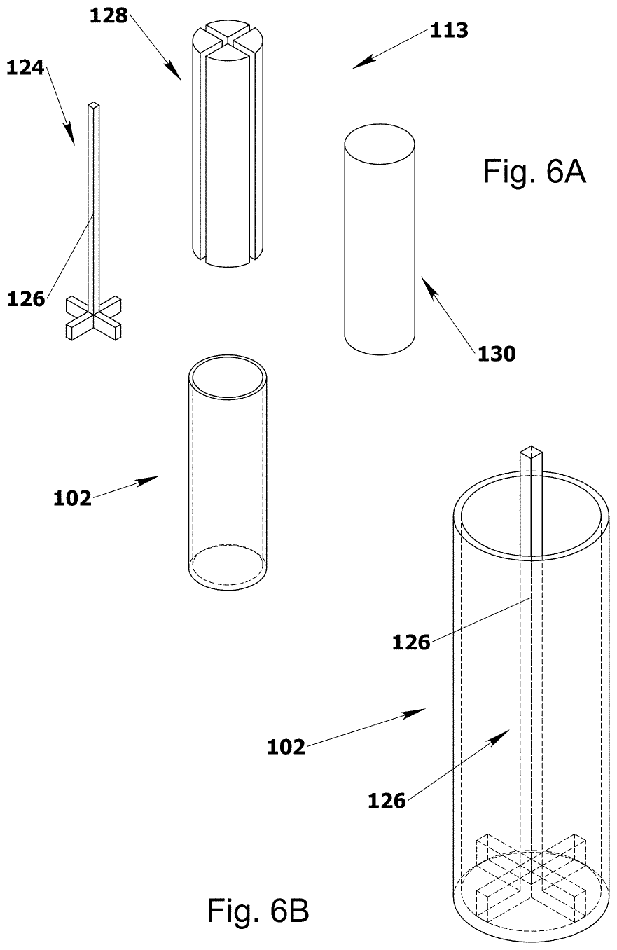

Reference is now made to FIGS. 6A and 6B, showing an example of assembly 113, including the constituents of form 102, fabricating module 110 and compacting module 112 of the segmental scaffolding fabricating machine 104 and fabricating module 120 and compacting module 122 of the structured payload substance pellets fabricating machine 114 of the industrial manufacture system 100 shown in FIG. 5, configured for essentially dry fabricating of a readily dissolvable cartridge complex. The example of assembly 113 of the embodiment of FIGS. 6A and 6B illustrates various features that may be interchangeable with elements of any other embodiment described in the specification.

In some embodiments, the industrial manufacture system comprises at least one stencil 124. In some embodiments, the exterior outline of least one stencil 124 essentially conforms and/or matches with a shape of axial partitions of the segmental scaffolding, extending transversally to and along the longitudinal centerline of the dissolvable cartridge complex. In some embodiments, at least one stencil 124 comprises mandrel 126. Mandrel 126 is configured for manipulating the distal part of stencil 124 within form 102.

In some embodiments, compacting mechanism 112 comprises pellet pressing piston array 128. The exterior outline of the pistons, in pellet pressing piston 128, essentially conforms and/or matches with exterior outline of a shape of the structured payload substance pellets. Pellet pressing piston array 128 is operationally connected to pressure exerting device 123, configured to compress the structured payload substance pellets, within form 102.

In some embodiment, compacting mechanism 112 further comprises segment pressing piston 130 operationally connected to pressure exerting device 123. Segment pressing piston 130 configured to compress at least one segment of the segmental scaffolding of the readily dissolvable cartridge complex within form 102.

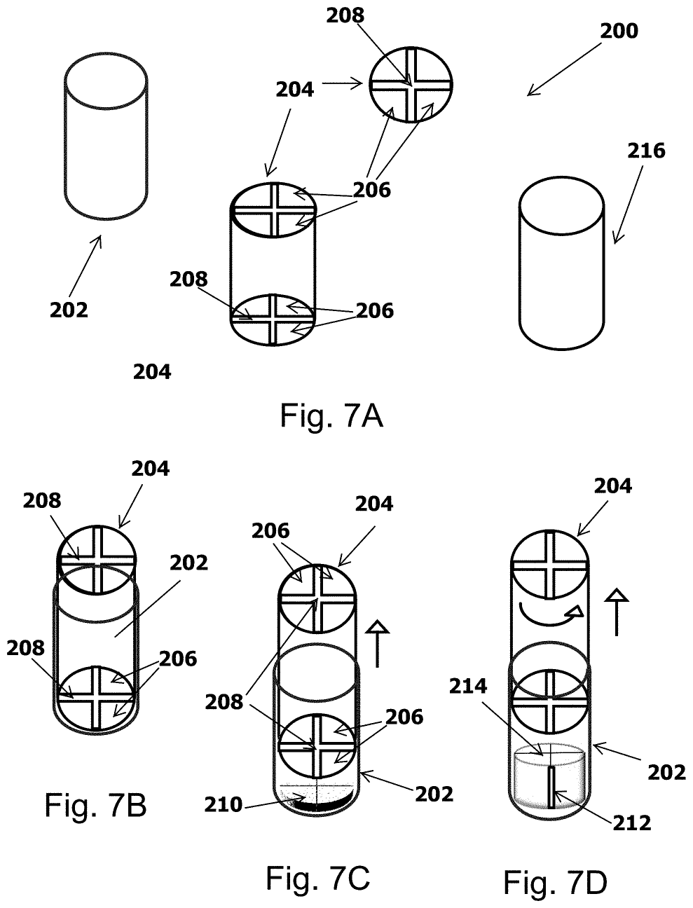

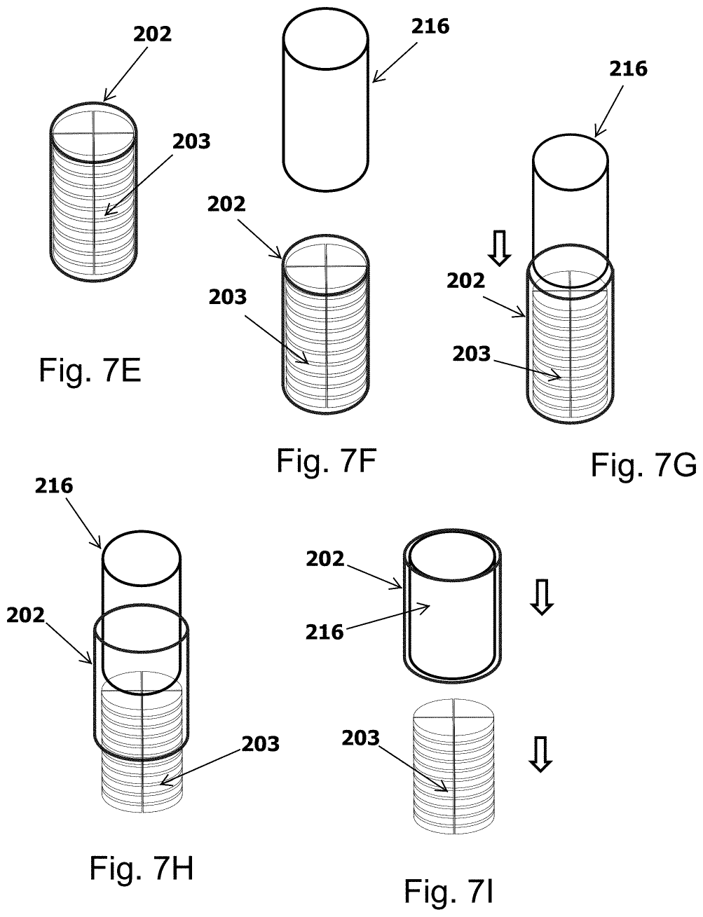

Reference is now made to FIG. 7A to 7I, showing another example of assembly 200, including the constituents of form 102, fabricating module 110 and compacting module 112 of the segmental scaffolding fabricating machine 104 and fabricating module 120 and compacting module 122 of the structured payload substance pellets fabricating machine 114 of the industrial manufacture system 100 shown in FIG. 5, configured for essentially dry fabricating of a readily dissolvable cartridge complex. The example of assembly 200 of the embodiment of FIG. 7A to 7I illustrates various features that may be interchangeable with elements of any other embodiment described in the specification.

In some embodiments, assembly 200 comprises form 202. Form 202 comprising an essentially hollow shape, in which an interior surface of form 202 essentially conforms and/or matches with a shape of dissolvable cartridge complex 203, such as dissolvable cartridge complex 10 shown in FIG. 1A to 3C and described hereinabove. It should be appreciated that the shape of the dissolvable cartridge complex, such as complex 10 shown in FIG. 1A to 3C and/or complex 203, is shown as cylindrical merely by a way of example, whereas numerous profiled shapes, such as rectangular, elliptical, quadratic, triangular, polygonal and/or structured are equally applicable.

In some embodiments, assembly 200 further comprises structured nozzle 204. The exterior outline of structured nozzle 204 essentially conforms and/or matches with exterior outline of dissolvable cartridge complex 203, whereas the shape of the interior outline of structured nozzle 204 essentially conforms and/or matches with exterior outline the structured payload substance pellets and with a shape of axial partitions of the segmental scaffolding, extending transversally to and along the longitudinal centerline of dissolvable cartridge complex 203.

In some embodiments, structured nozzle 204 comprises a plurality of payload substance nozzles 206. Payload substance nozzles 206 are configured for administrating the payload substance into form 202, thereby forming a plurality of structured payload substance pellets. In some embodiments, structured nozzle 204 comprises effervescent material nozzle 208. Effervescent material nozzle 208 is configured for administrating the effervescent material into form 202, thereby forming the axial partitions between the pellets, as well as the radial partitions between the segments.

In some embodiments, structured nozzle 204 with plurality payload substance nozzles 206 and effervescent material nozzle 208 is insertable into form 202. Payload substance nozzles 206 and effervescent material nozzle 208 are implementable for administrating, optimally concomitantly, the payload substance and the effervescent material, while vertically translating structured nozzle 204 within form 202 in an upward direction, thereby forming first segment 210 of dissolvable cartridge complex 203 within form 202.

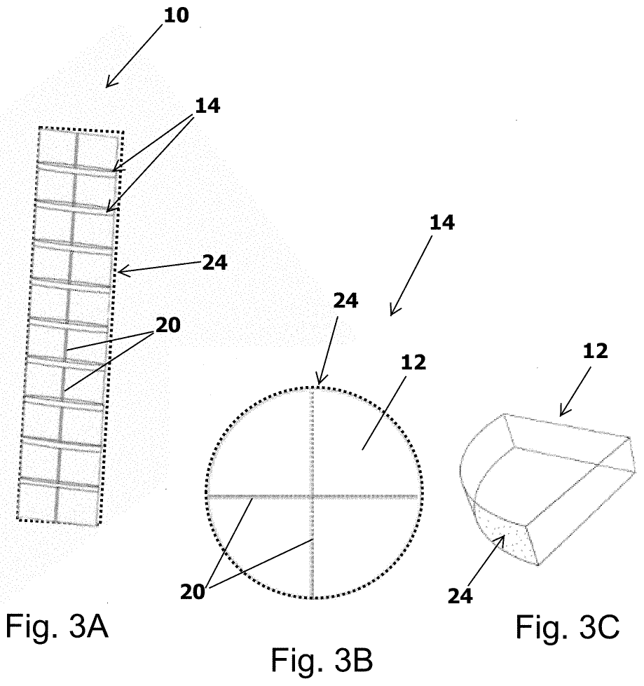

In some embodiments, structured nozzle 204 is rotatable about longitudinal centerline of structured nozzle 204 while additionally filling from effervescent material nozzle 208 a layer of effervescent material above constructed segment 210, thereby providing material for radial partition 212, such as radial partitions 16 shown in FIG. 3A, between the segments of dissolvable cartridge complex 203.

In some embodiments, upon administering the effervescent material for radial partition 214, payload substance nozzles 206 and effervescent material nozzle 208 are implementable for administrating, optimally concomitantly, the payload substance and the effervescent material, while vertically translating structured nozzle 204 within form 202 in an upward direction, thereby forming the next segment of dissolvable cartridge complex 203.

In some embodiments, assembly 200 further comprises pressing piston 216. Pressing piston 216 is insertable into form 203 once structured nozzle 204 is withdrawn therefrom. The exterior outline of pressing piston 216, essentially conforms and/or matches with exterior outline of a shape of the readily dissolvable cartridge complex. Pressing piston 210 is configured to compact readily dissolvable cartridge complex 203 within form 202 and/or for ejecting readily dissolvable cartridge complex 203 from form 202.

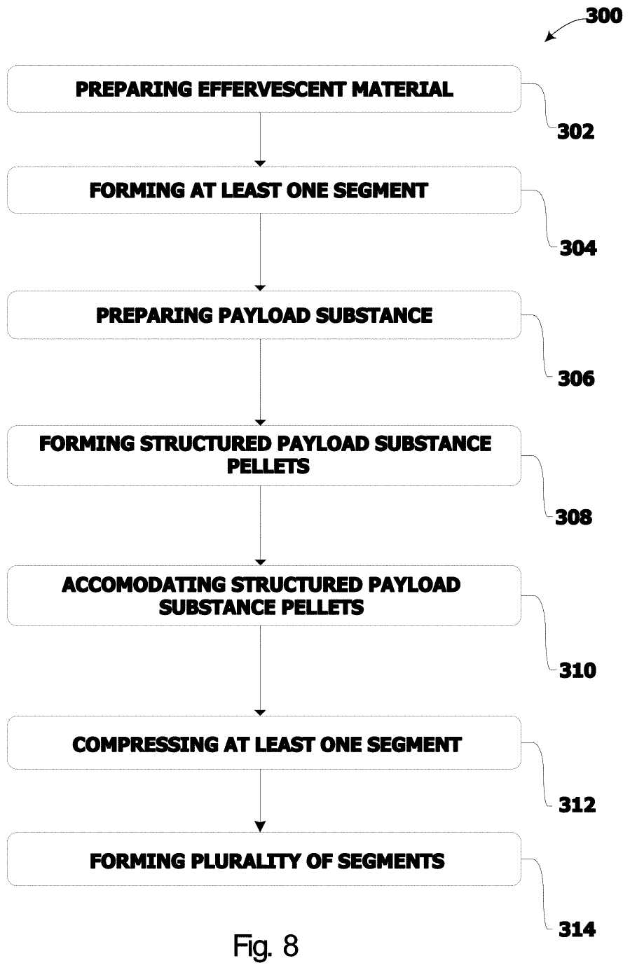

In accordance with some embodiments of the present invention, reference is now made FIG. 8 showing a flowchart of industrial process 300 of fabricating a readily dissolvable cartridge complex. The process of the embodiment of FIG. 8 illustrates various features that may be interchangeable with elements of any other embodiment described in the specification.

In some embodiments, process 300 commences with step 302 of preparing an effervescent material readily dissolvable in an aqueous solution. In some embodiments, process 300 comprises step 304 of forming at least one segment of a segmental scaffolding from the effervescent material. Step 304 typically further comprises forming at least on radial partition of at least one segment of the segmental scaffolding, extending about the longitudinal centerline of the dissolvable cartridge complex.

In some embodiments, step 304 comprises forming a plurality of axial partitions within at least one segment of the segmental scaffolding, extending transversally to and along the longitudinal centerline of the dissolvable cartridge complex. In some embodiments, step 304 further comprises forming a plurality of compartments formed in-between the axial partitions, in each one from the plurality of the segments of the segmental scaffolding.

In some embodiments, process 300 includes step 306 of preparing a payload substance. In some embodiments, process 300 further comprises step 308 of forming a plurality of structured payload substance pellets. In some preferred embodiments, step 304 of forming at least one segment of a segmental scaffolding from the effervescent material is performed concomitantly with step 308 of forming a plurality of structured payload substance pellets, by a plurality of nozzles.

In some embodiments, process 300 includes step 310 of accommodating the structured payload substance pellets in the compartments of the segmental scaffolding. In some embodiments, process 300 further comprises step 312 of compressing at least one segment of the segmental scaffolding of the readily dissolvable cartridge complex.

In some embodiments, process 300 includes a step of deploying at least one stencil, in which an exterior outline of at least one stencil essentially conforms and/or matches with a shape of axial partitions of the segmental scaffolding, extending transversally to and along the longitudinal centerline of the dissolvable cartridge complex. In some embodiments, process 300 includes step of deploying at least one stencil, in which an exterior outline of at least one stencil essentially conforms and/or matches with a shape of the structured payload substance pellets.

In some embodiments, process 300 includes a step of saturating the payload substance and the effervescent material, with a predefined amount of saturating substrate, thereby conferring the payload substance and the effervescent material a semi-liquefied consistency. The term semi-liquefied consistency comprises a slurry and/or sludge like consistency. In some embodiments, process 300 includes a step of controllably delivering the payload substance and the effervescent material, into a predetermined location within the form.

In some embodiments, process 300 comprises step 314 of iteratively forming a plurality of segments of the readily dissolvable cartridge complex separated by the radial partitions.

In some embodiments, process 300 further includes a step of furnishing the readily dissolvable cartridge complex with a coating configured to absorb and/or neutralize undesired additives or impurities in water. In some embodiments, process 300 includes further a step of furnishing the readily dissolvable cartridge complex with a coating comprising water softening agent, including natrium chloride.

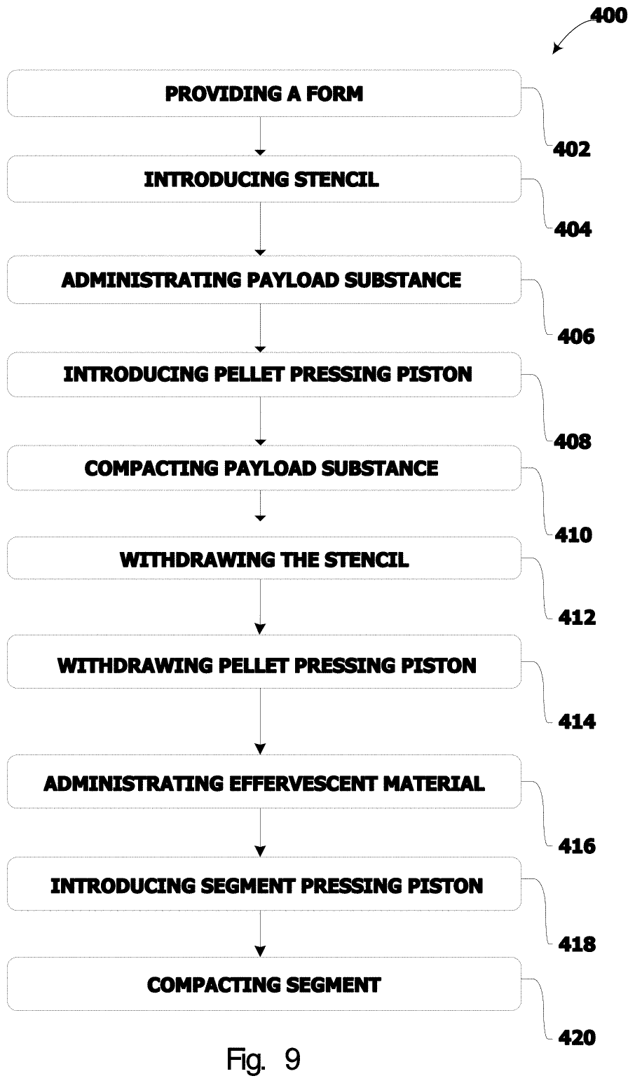

In accordance with some embodiments of the present invention, reference is now made FIG. 9 showing a flowchart of industrial process 400 of essentially dry fabricating of the readily dissolvable cartridge. The process of the embodiment of FIG. 9 illustrates various features that may be interchangeable with elements of any other embodiment described in the specification.

In some embodiments, process 400 commences with step 402 of providing a form, such as form 102 shown in FIGS. 6A and 6B. In some embodiments, process 400 further comprises step 404 of introducing a stencil, such as stencil 124 shown in FIGS. 6A and 6B, into the form.

In some embodiments, process 400 further includes step 406 of administrating the payload substance into the form. Step 406 typically comprises flattening and/or leveling the payload substance using vibration and/or a dedicated tool and/or conveying mini screws and/or air pressure and/or mechanical pressure. In some embodiments, process 400 further comprises step 408 of introducing a pellet pressing piston, such as pellet pressing piston array 128 shown in FIGS. 6A and 6B, into the form. The pellet pressing piston essentially the exterior outline of the structured payload substance pellet.

In some embodiments, process 400 further comprises step 410 of compacting the payload substance, thereby forming the plurality of structured payload substance pellets, within one segment. In some embodiments, step 406 of administrating the payload substance into the form as well as step 410 of compacting the payload substance are performed in combination and a priori, so that the plurality of structured payload substance pellets are pre-pressed ahead, so that already compacted structured payload substance pellets are administered into the form. In some embodiments, process 400 further includes step 412 of withdrawing the stencil from the form.

In some embodiments, process 400 further comprises step 414 of withdrawing the pellet pressing piston from the form. In some embodiments, process 400 further includes step 416 of administrating an effervescent material into the form, thereby filling the void spaces between the structured payload substance pellets thereby forming the axial partitions between the pellets, as well as additionally filling a layer above the structured payload substance pellets in the constructed segment, thereby providing material for the radial partition, such as radial partitions 16 shown in FIG. 3A, between the segments. In some embodiments, step 416 further comprises step of flattening and/or leveling the effervescent material using vibration and/or a dedicated tool and/or conveying mini screws and/or air pressure and/or mechanical pressure.

In some embodiments, process 400 further comprises step 418 of introducing a segment pressing piston, such as segment pressing piston 130 shown in FIG. 6A, into the form. The segment pressing piston assumes essentially the entire cross-sectional area of the form. In some embodiments process 400 includes step 420 of compacting, thereby forming a segment within the readily dissolvable cartridge complex. In some embodiments, process 400 yet further comprises a step of withdrawing the segment pressing piston from the form. In some embodiments, process 400 ultimately includes an iterative step of repeating steps 404 to 420.

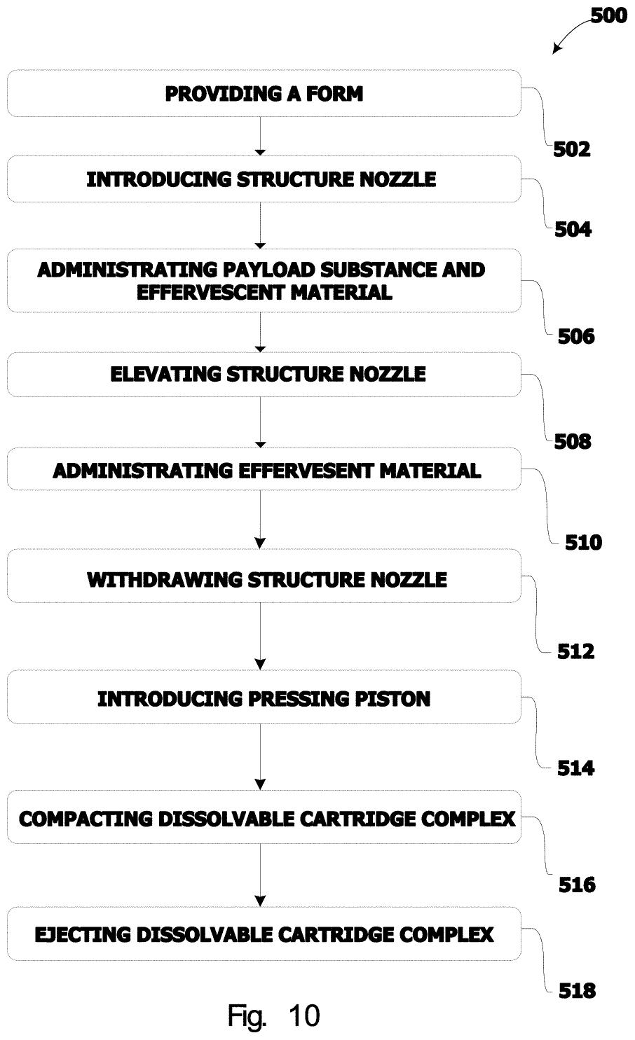

In accordance with another embodiment of the present invention, reference is now made FIG. 10 showing a flowchart of industrial process 500 of essentially dry fabricating of the readily dissolvable cartridge. The process of the embodiment of FIG. 10 illustrates various features that may be interchangeable with elements of any other embodiment described in the specification.

In some embodiments, process 500 commences with step 502 of providing a form, such as form 202 shown in FIG. 7A to 7I. In some embodiments, process 500 further comprises step 504 of introducing a structured nozzle, such as structured nozzle 204 shown in FIG. 7A to 7I, into the form. The structured nozzle comprises a plurality of dedicated payload substance nozzles and an effervescent material nozzle.

In some embodiments, process 500 further includes step 506 of administrating, optionally simultaneously, the payload substance and the effervescent material into the form, via the structured nozzle. Step 506 typically comprises flattening and/or leveling the payload substance using vibration and/or a dedicated tool and/or conveying mini screws and/or air pressure and/or mechanical pressure. In some embodiments, process 500 further comprises step 508 of elevating the structured nozzle by axial translation of the structured nozzle within the form in an upward direction, thereby forming a segment of the readily dissolvable cartridge complex.

In some embodiments, process 500 further comprises step 510 of administrating a layer of effervescent material, optionally from the effervescent material nozzle, while elevating by axial translation of the structured nozzle within the form in an upward direction and optionally rotating the structured nozzle, thereby providing material for the radial partition, such as radial partition 16 shown in FIG. 3A, between the segments. Step 510 typically comprises flattening and/or leveling the effervescent material. In some embodiments, process 500 ultimately includes an iterative step of repeating steps 506 to 510.

In some embodiments, process 500 further comprises step 512 of withdrawing the structured nozzle from the form once the entire readily dissolvable cartridge complex and/or a single segment of the readily dissolvable cartridge complex is formed. In some embodiments, process 500 further comprises step 514 of introducing a pressing piston, such as segment pressing piston 130 shown in FIGS. 6A and 6B, into the form. The segment pressing piston typically assumes essentially the entire cross-sectional area of the form.

In some embodiments, process 500 further comprises step 516 of compacting the readily dissolvable cartridge complex. In some embodiments, process 500 further concluded with step 518 of ejecting the readily dissolvable cartridge complex from the form.

It will be appreciated by persons skilled in the art of the invention that various features and/or elements elaborated in the context of a specific embodiment described hereinabove and/or referenced herein and/or illustrated by a particular example in a certain drawing enclosed hereto, whether method, system, device or product, is/are interchangeable with features and/or elements of any other embodiment described in the specification and/or shown in the drawings.

Moreover, skilled persons would appreciate that the present invention is not limited by what has been particularly shown and described hereinabove. Rather the scope of the invention is defined by the claims which follow:

1. An industrial process of fabricating a readily dissolvable cartridge complex comprises:
(a) preparing an effervescent material readily dissolvable in an aqueous solution;
(b) forming at least one segment of a segmental scaffolding from said effervescent material comprising:
(I) forming at least one radial partition of said at least one segment of said segmental scaffolding, extending about said longitudinal centerline of said dissolvable cartridge complex;
(II) forming a plurality of axial partitions within said at least one segment of said segmental scaffolding, extending transversally to and along said longitudinal centerline of said dissolvable cartridge complex;
(Ill) forming a plurality of compartments formed in-between said axial partitions, in each one of said plurality of said segments of said segmental scaffolding;
(c) preparing a payload substance;
(d) forming a plurality of structured payload substance pellets;
(e) accommodating said structured payload substance pellets in said compartments of said segmental scaffolding;
(f) compressing at least one segment of said segmental scaffolding of said readily dissolvable cartridge complex;
(g) iteratively forming a plurality of segments of said readily dissolvable cartridge complex separated by said radial partitions.

2. The industrial process, as in claim 1, further comprises furnishing said readily dissolvable cartridge complex with a coating configured to absorb and/or neutralize undesired additives or impurities in water.

3. The industrial process, as in claim 1, further comprises a furnishing said readily dissolvable cartridge complex with a coating comprising water softening agent, including natrium chloride.

4. The industrial process, as in claim 1, further comprises controllably saturating at least one member selected from the group consisting of: said payload substance and said effervescent material, with a predefined amount of saturating substrate, thereby conferring to said at least one member a semi-liquefied consistency.

5. The industrial process, as in claim 1, further comprises:
(a) controllably saturating said payload substance and said effervescent material, thereby conferring thereto a semi-liquefied consistency, and
(b) controllably delivering said payload substance and said effervescent material, into a predetermined location within said form.

6. The industrial process, as in claim 1, further comprises deploying at least one stencil, wherein an exterior outline of said at least one stencil essentially matches at least one shape selected from the group consisting of:
(a) a shape of axial partitions of said segmental scaffolding, extending transversally to and along said longitudinal centerline of said dissolvable cartridge complex;
(b) a shape of said structured payload substance pellets.

7. The industrial process as in claim 1, wherein said compressing comprises essentially matches with exterior outline of a shape of said structured payload substance pellets.

8. An industrial manufacture system for fabricating a readily dissolvable cartridge complex comprises:
(a) at least one form, comprising an essentially hollow shape, wherein an interior surface of said at least one form essentially matches with a shape of said readily dissolvable cartridge complex;
(b) a segmental scaffolding fabricating machine comprising:
(I) an effervescent material reservoir, containing an effervescent material readily dissolvable in an aqueous solution;
(II) a dossing module connected to said reservoir of effervescent material, configured to receive said effervescent material from said reservoir of effervescent material and to portion a predetermined amount of said effervescent material;
(III) a fabricating module comprising at least one nozzle, configured to dispose said effervescent material in a predefined structured arrangement within said form, thereby forming a plurality of compartments, within at least one segment of said segmental scaffolding;
(IV) a compacting mechanism, comprising at least one pressing piston operationally connected to a pressure exerting device, configured to compress at least one segment of said segmental scaffolding of said readily dissolvable cartridge complex within said form;
(c) a structured payload substance pellets fabricating machine comprising:
(I) a payload substance reservoir, containing a payload substance;
(II) a dossing module connected to said reservoir of payload substance, configured to receive said payload substance from said reservoir of payload substance and to portion a predetermined amount of said payload substance;
(III) a fabricating module comprising at least one nozzle, configured to dispose said payload substance in a predefined structured arrangement within said form, thereby forming a plurality of structured payload substance pellets within said compartments in at least one segment of said segmental scaffolding;
(IV) a compacting mechanism, comprising at least one pressing piston operationally connected to a pressure exerting device, configured to compress said structured payload substance pellets within said compartments, thereby accommodating said structured payload substance pellets in said compartments, formed within at least one segment of said segmental scaffolding of said readily dissolvable cartridge complex, within said form.

9. The industrial manufacture system, as in claim 8, further comprises a saturating module, configured for controllably saturate at least one member selected from the group consisting of: said payload substance and said effervescent material, with a predefined amount of saturating substrate, thereby conferring to said at least one member a semi-liquefied consistency.

10. The industrial manufacture system, as in claim 8, further comprises a pulverizator, configured for furnishing said readily dissolvable cartridge complex with a coating configured to absorb and/or neutralize undesired additives or impurities in water.

11. The industrial manufacture system as, in claim 8, further comprises at least one stencil, wherein an exterior outline of said at least one stencil essentially matches with at least one shape selected from the group consisting of:
(a) a shape of axial partitions of said segmental scaffolding, extending transversally to and along said longitudinal centerline of said dissolvable cartridge complex;
(b) a shape of said structured payload substance pellets.

12. The industrial manufacture system as in claim 8, wherein said compacting mechanism of said structured payload substance pellets fabricating machine comprises a plurality of pressing pistons, wherein an exterior outline of said pistons essentially matches with exterior outline of a shape of said structured payload substance pellets.

13. A readily dissolvable cartridge complex comprises:
(a) a segmental scaffolding comprising an effervescent material readily dissolvable in an aqueous solution;
(b) a plurality of radial partitions of said segmental scaffolding, arranged in tandem, extending about said longitudinal centerline of said dissolvable cartridge complex;
(c) a plurality of segments of said dissolvable cartridge complex, separated by said radial partitions and formed in-between said radial partitions;
(d) a plurality of axial partitions of said segmental scaffolding, extending transversally to and along said longitudinal centerline of said dissolvable cartridge complex, dividing each one of said plurality of said segments of said dissolvable cartridge complex into a plurality of sectors;
(e) a plurality of compartments formed in-between said axial partitions within said sectors, in each one of said plurality of said segments of said dissolvable cartridge complex;
(f) a plurality of structured payload pellets, accommodated in said compartments.

14. The readily dissolvable cartridge complex, as in claim 13, further comprises a coating configured to absorb and/or neutralize undesired additives or impurities in water.

15. The readily dissolvable cartridge complex, as in claim 13, further comprises a coating comprising a water softening agent.

16. The readily dissolvable cartridge complex as in claim 13, further comprises a coating including natrium chloride.
